(19) United States
(12) Patent Application Publication
(10) Pub. No.: US 2020/0345794 A1
(43) Pub. Date: Nov. 5, 2020

(54) COMPOSITION FOR IMPROVING THE WELL-BEING OF ANIMALS

(71) Applicant: NOLIVADE, CHANGÉ (FR)

(72) Inventors: Michel LAYUS, PLEUMEUR BODOU (FR); Alexandre BRAME, RENNES (FR); Stéphane THEBAULT, SAINT GERMAIN LE FOUILLOUX (FR)

(73) Assignee: NOLIVADE, CHANGÉ (FR)

(21) Appl. No.: 16/762,260

(22) PCT Filed: Nov. 8, 2018
(86) PCT No.: PCT/EP2018/080666

§ 371 Date: May 7, 2020

(30) Foreign Application Priority Data
Nov. 8, 2017 (FR) 1760495

(51) Int. Cl. A61K 35/742; A61P 1/00; A61P 31/04; A61K 9/00; A61K 35/747; A01N 63/22; A01N 63/20

(52) U.S. Cl. CPC A61K 35/742; A61P 1/00; A61P 31/04; A61K 9/0053; A61K 2035/115; A61K 35/747; A01N 63/22; A01N 63/20; A61K 9/007

(57) ABSTRACT

A method for the prevention or treatment of pathogens affecting the health of livestock animals, which includes the administration to the livestock animals a composition having at least one bacterial strain of the genus Bacillus and at least one lactic bacterium. Also, a kit for the prevention of pathogenic infections affecting the health and well-being of livestock animals, which includes the composition.

### FIELD

The invention relates to a composition aimed at improving the well-being of animals, in particular livestock animals.

### BACKGROUND

Health problems on farms are often the result of a combination of several pathogens. These can be present in different microbial ecosystems: the environment (air, organic or inorganic surfaces), in the animal's internal system (digestive or respiratory system) or on the animal (skin).

In the various types of terrestrial animal farms, there are always risk phases where the microbial ecosystems evolve strongly, such as a phase of reimplantation of an external flora after disinfection, the arrival of animals on a farm, the first moments of life of newborns, the arrival of new individuals in a group of already existing animals, feed transitions, the phase of reimplantation of an internal flora after the use of antibiotics, etc.

Thus, it is necessary to control this environment in order to maintain the health status of farms and to safeguard animal well-being.

Antibiotic treatment strategies have been in use for a very long time but pose two major problems: firstly, resistance generated by the overly regular use of antibiotics, and secondly, a public health problem through the consumption of animal feed containing many antibiotics.

It is therefore necessary to find alternatives to antibiotics.

A more recent approach has been developed which is aimed at the use of bacterial flora to regulate pathogenic flora found in farms. For example, applications in the environment such as in application EP0852114, where non-pathogenic bacteria are used to propose a process for systematic disinfection of buildings intended for the rearing of a herd.

Other approaches are being used and are aimed at treating animals directly through the skin with non-pathogenic bacteria. Examples include, in particular, application CN104307012, which proposes deodorant and sanitizing compositions that can be sprayed on poultry, or application CN103773721, which proposes probiotic solutions that limit the risk of diarrhea in newborns, examples of application being a spray on the mother's udder.

However, despite these different methods, the health results do not appear to be satisfactory.

Therefore, there is a need to find a solution to maintain a healthy environment for the rearing of terrestrial animals in order to promote the health and thus well-being of these animals, which will in turn promote livestock animal productivity.

### SUMMARY

The object of the invention is to overcome the above-mentioned disadvantages.

Another object of the invention is to provide a new and effective method for maintaining the health and well-being of terrestrial livestock animals.

Another object of the invention is to provide a kit that can be used directly by farmers.

The invention therefore relates to a bacterial composition comprising at least one bacterial strain of the genus Bacillus, in particular Bacillus subtilis, and at least one strain of lactic bacteria, in particular at least one strain of Lactococcus lactis, for use in the context of the prevention or treatment of pathogenic infections affecting the health of terrestrial livestock animals,

said composition being:

a. ingested or inhaled by said terrestrial livestock animals, and

b. either

b.1. spread within the rearing environment of said terrestrial livestock animals,

b.2. applied to said terrestrial livestock animals,

b.3. spread within the rearing environment of said terrestrial livestock animals and applied to said terrestrial livestock animals.

### BRIEF DESCRIPTION OF DRAWINGS

FIG. 1 is a graph showing the corrected consumption index (CCI) results for trial and control buildings. The x-axis differentiates between the results of the trial batches (left) and control batches (right). The y-axis represents the CCI values. For all the trial batches, identified by the black dots in the left column, a synthetic boxplot is shown in order to display means, medians, quantiles and confidence indices. The same is true for the control batches in the left column. A: invention; B: controls.

FIG. 2 is a graph showing the difference of corrected consumption index (CCI) results between trial and control buildings on the same farm. The y-axis represents the CCI values. For all the farms, identified by the black dots, a synthetic boxplot is shown to display means, medians, quantiles and confidence indices.

FIG. 3 is a graph showing the results of individual weighing of animals at 7 days of age from all farms in the trial and control buildings. The x-axis differentiates between the results of the trial batches (left—A.) and control batches (right—B.). The y-axis represents the weight values in kg. A synthetic boxplot shows the values of the means, medians, quantiles and confidence indices for all animals from the trial batches (left) and control batches (right).

FIG. 4 is a histogram showing the results of burned tarsi obtained at the slaughterhouse for female chickens. The abscissa differentiates between control batches (light grey on the left) and trial batches (dark grey on the right). The y-axis represents the percentage of burned tarsi. The graphs show the value of the medians of the two batches and their confidence interval.

FIG. 5 is a histogram showing the results of burned tarsi obtained at the slaughterhouse for male chickens. The abscissa differentiates between control batches (light grey on the left) and trial batches (dark grey on the right). The y-axis represents the percentage of burned tarsi. The graphs show the value of the medians of the two batches and their confidence interval.

FIG. 6 is a histogram showing the antibiotic consumption results in ppm of antibiotic per kg body weight. The x-axis differentiates between control batches (light grey on the left) and trial batches (dark grey on the right). The y-axis represents the antibiotic consumption value. The graphs show the value of the medians for the two batches and their confidence interval.

FIG. 7 is a histogram showing the number of days of cumulative antibiotic treatments over all trial or control batches. The x-axis differentiates between treatments carried out for problems related to lameness (A.) or treatments carried out in the first 10 days of life of the animals (start B.). The y-axis shows the number of days of antibiotic treatments. The results for the trial batches are differentiated in light grey and the results for the control batches in dark grey.

FIG. 8 is a histogram showing the mean weight results obtained at the slaughterhouse for male chickens. The x-axis differentiates between control batches (light grey on the left) and trial batches (dark grey on the right). The y-axis shows the mean weight of the animals. The graphs show the value of the medians of the two batches and their confidence intervals.

FIG. 9 is a histogram showing the mean weight results obtained at the slaughterhouse for female chickens. The x-axis differentiates between control batches (light grey on the left) and trial (dark grey on the right) batches. The y-axis represents the mean weight of the animals. The graphs show the value of the medians of the two batches and their confidence intervals.

FIG. 10 represents the modeling of the evolution of cumulative mortality in female chickens. The y-axis shows the days of life of the animals and the y-axis shows the percentage of cumulative deaths. The light grey (top) shows the model for the control batches and the dark grey (bottom) shows the model for the trial batches.

FIG. 11 shows the modeling of the risk of diarrhea occurrence in a pen according to the protocol followed. The x-axis shows the days of life of the piglets in relation to the farrowing date (0). The y-axis shows the probability of at least one piglet in a pen having diarrhea. The three grouped curves at the top show the diarrhea probability models for batches 1 to 3, and the isolated curve at the bottom shows that for batch 4.

FIG. 12 shows the modelling of the mortality risk of a piglet according to the protocol followed. The x-axis shows the days of life of the piglets in relation to the farrowing date (0). The y-axis shows the probability that a piglet will die. The three grouped curves at the top show the diarrhea probability models for batches 1 to 3, and the isolated curve at the bottom shows that for batch 4.

FIG. 13 is a graph showing the number of piglet pens with normal feces from 120 pens with shampooing and 120 pens without shampooing. The y-axis shows the days of life of the piglets and the y-axis shows the number of pens with normal feces. The light grey shows the pens that were not been shampooed before receiving the flora and the black shows the pens that were shampooed before receiving the flora.

### DETAILED DESCRIPTION

The invention is based on the surprising finding by the inventors that a mixture of at least one bacterial strain of the genus Bacillus subtilis and at least one strain of the genus Lactococcus lactis, non-pathogenic bacteria, act synergistically on the animal health of terrestrial animals when said mixture is both ingested or inhaled by the animals and either spread within the rearing environment or applied directly to the animal, or both. In the invention, the spreading is assimilated to dispersion; both terms may be used uniformly in the invention.

The inventors have demonstrated a synergistic protective effect against pathogens when the bacteria described in the invention (Bacillus and Lactococcus) are both present in the digestive or respiratory tract and in the rearing environment and/or on the animal.

In the invention, a bacterial strain is understood to mean a set of individuals resulting from successive transplantations of a bacterial colony. A bacterial strain is a part of a bacterial species different from other bacteria of the same species by a minor but identifiable difference.

In the invention, pathogenic infections are defined as infections by the following microorganisms: Salmonella, Staphylococci, Streptococci, Clostridium, Listeria, Arcanobacterium, Mycoplasma, Klebsiella, Pasteurella, Bordetella, Campylobacter, Brachyspira, Escherichia coli and/or responsible for the following pathologies: enteritis, salmonellosis, ulcers, infectious lameness, rhinitis, mastitis, epidermitis, necrosis, pasteurellosis, bordetellosis, listeriosis.

It is particularly advantageous in the invention that the above-mentioned composition comprises at least two strains of Bacillus, in particular at least two different strains of Bacillus and at least one strain of lactic bacteria, in particular Lactococcus lactis.

It is even more advantageous in the invention that the above-mentioned composition comprises at least three strains of Bacillus, and in particular at least three different strains of Bacillus, and at least one strain of lactic bacteria, in particular Lactococcus lactis.

It is even more advantageous in the invention that the above-mentioned composition comprises at least four strains of Bacillus, and in particular at least four different strains of Bacillus and at least one strain of lactic bacteria, in particular Lactococcus lactis.

In the invention a distinction is made between an application in the environment (E), with reference to spreading in the environment, an external application on the animal (A), with reference to application on the animal, and an internal application (I), with reference to ingestion or inhalation.

Application E makes it possible to control and/or direct the bacterial flora of the terrestrial livestock animal environment by reducing pathogen pressure. It can be done by nebulization or spraying of livestock buildings, incorporation into bedding, etc. Less contamination of the animals is observed because there are fewer pathogenic outbreaks. Such an application acts on living or surviving pathogens in the environment. The methods of spreading in the environment are spraying, nebulization, covering surfaces with foams or dusting with powder.

Spreading is generally done on livestock surfaces, mainly on surfaces in direct or potential contact with animals, preferably on surfaces that have been cleaned and disinfected, after waiting for the disinfectants to wear off.

Spreading in the environment is carried out at least once after a sanitary vacuum, after a cleaning/disinfection protocol or before the arrival of the animals. It is particularly advantageous to carry out a repeated application every week.

Application A makes it possible to control or direct the animal's microbism by decreasing the pathogen pressure. The application can be done by nebulization, or spraying or soaking of the terrestrial animal. This generates less contamination of the animals because there are fewer pathogenic outbreaks directly on the animal.

Application to the animal may be made by spraying, nebulization, soaking, foot bath, or foam, depending on the area of the animal on which it is desired to deposit the composition according to the invention.

It is advantageous to apply the composition at least once to the animal as early as possible in the animal's life or upon arrival in a new environment. Preferably, the application should be repeated weekly.

Application I makes it possible to control and/or direct the digestive microbism by decreasing the pathogenic pressure and/or by stimulating the natural defenses of the animals. The application can be implemented via drinking water, feed or any means allowing the animal to ingest the composition of the invention. In this way, the composition according to the invention directly acts on the digestive health of terrestrial livestock animals, thereby limiting the entry of other pathogens.

Application I may also be an application by inhalation, nebulization or by spraying into the airways.

Application I should be started as early as possible, before the start of the health risk phase and for the duration of the health risk period. It is recommended to repeat the application at least every 10 days, at best every day.

In the invention, terrestrial livestock animals are understood to mean all animals that are not aquatic and that can be used directly or indirectly for human or animal nutrition. This concerns in particular cattle, sheep, pigs, goats, rabbits, horses, insects, but also farmed birds such as hens, ducks, geese, turkeys, quails and other farmed gallinaceous animals.

Advantageously, the invention relates to a composition for its aforementioned use, wherein the bacterial composition comprises:
at least one of the following three strains of Bacillus subtilis: NOL01, NOL02, NOL03, said strains being deposited, respectively, at the CNCM under the numbers CNCM I-4606, CNCM I-5043 and CNCM I-4607, and
at least the strain of lactic bacteria: Lactococcus lactis spp lactis 1 strain NOL11, said strain being deposited at the CNCM under the number CNCM I-4609.

The invention relates to the use of at least one strain selected from the strains NOL01, NOL02 and NOL03 and at least the strain NOL11. Also, the invention covers the following 7 combinations:

NOL01 and NOL11,

NOL02 and NOL11,

NOL03 and NOL11,

NOL01, NOL02 and NOL11,

NOL01, NOL03 and NOL11,

NOL02, NOL03 and NOL11, and

NOL01, NOL02, NOL03 and NOL11.

All of these strains were deposited at the National Collection of Microorganisms (CNCM) at the Pasteur Institute in Paris in accordance with the Budapest Treaty.

More advantageously, the invention relates to a composition for its aforementioned use, said composition comprising from 10⁴ to 10¹¹ bacterial colonies of Bacillus and from 10⁴ to 10¹¹ bacterial colonies of lactic bacteria, the bacterial colonies being in grams or milliliters of composition.

In other words, in this advantageous embodiment, if the composition according to the invention is in liquid form, said composition will comprise from 10⁴ to 10¹¹ bacterial colonies of Bacillus per mL of composition, this being the case for each of the strains when the composition comprises at least two strains, and from 10⁴ to 10¹¹ bacterial colonies of lactic bacteria per mL of composition.

If, on the other hand, the composition according to the invention is dehydrated or non-aqueous, the said composition will comprise from 10⁴ to 10¹¹ bacterial colonies of Bacillus per g of composition, this being the case for each of the strains when the composition comprises at least two strains, and from 10⁴ to 10¹¹ bacterial colonies of lactic bacteria per g of composition.

In the invention, from 10⁴ to 10¹¹ bacterial colonies means: about 10⁴, about 5.10⁴, about 10⁵, about 5.10⁵, about 10⁶, about 5.10⁶, about 10⁷, about 5.10⁷, about 10⁸, about 5.10⁸, about 10⁹, about 5.10⁹, about 10¹⁰, about 5.10¹⁰ or about 10¹¹ bacterial colonies.

Bacterial colonies are defined by mL (or grams) of bacterial culture. The person in the field easily knows how to determine this number of bacteria, either by manual counting (using a Malassez slide) or by using an automatic cell counter, or by dilution and then agar plating and counting of colonies.

Advantageously, 10⁵ to 10¹⁰ bacterial colonies per individual will be used for an application in poultry, 10⁵ to 10¹⁰ bacterial colonies per individual for an application in piglets, and 10⁵ to 10¹¹ bacterial colonies per individual for an application in pigs.

Concerning the beneficial dose for ingestion, this is 10⁵ to 10¹⁰ bacterial colonies per animal per application.

For the environment, it is advantageous to use 10⁵ to 10¹¹ bacterial colonies per m² of surface or environment to be treated.

Even more advantageously, the invention relates to a composition for use as defined above, wherein said at least one strain of Bacillus is in sporulated and/or vegetative form.

Lactic bacteria are always in vegetative form. Thus, the composition according to the invention comprises at least one strain of Bacillus in sporulated form and at least one strain of lactic bacteria in vegetative form, or comprises at least one strain of Bacillus in vegetative form and at least one strain of lactic bacteria in vegetative form.

Even more advantageously, the invention relates to a composition for use as defined above, wherein said inhaled or ingested composition and said composition spread in the rearing environment of said terrestrial livestock animals and/or applied to said terrestrial livestock animals, are used simultaneously, separately or staggered over time.

It is not necessary for treatments ingested or inhaled and treatments spread within the environment or applied to the animal to be simultaneous, i.e. applied at the same time. It is possible to separate the applications over time, for example to start treating the environment of the animal, and then, once the animal is introduced into the environment, to have it ingest the composition according to the invention.

More advantageously, the invention relates to a composition for the aforementioned use, wherein said composition is spread and/or applied prior to ingestion or inhalation by said terrestrial livestock animals.

In the case of a treatment prior to ingestion or inhalation of the composition according to the invention, the spreading in the environment is carried out according to the doses as defined above.

Advantageously, the invention relates to a composition for its aforementioned use, wherein said composition consists essentially of the four bacterial strains from the group consisting of the three strains NOL01, NOL02, NOL03, said strains being deposited, respectively, at the CNCM under the number CNCM I-4606, CNCM I-5043 and CNCM I-4607, and the strain Lactococcus lactis spp lactis 1 strain NOL11, said strain being deposited at the CNCM under the number CNCM I-4609.

This combination is particularly advantageous and is described in the examples.

It is particularly advantageous that the composition comprises the three strains of Bacillus subtilis bacteria mentioned above in vegetative or sporulated form, and the lactic bacterium strain mentioned above in vegetative form.

Even more advantageously, the invention relates to a composition for its use as defined above, wherein said composition is spread per m² at a rate of 10⁵ to 10¹¹ bacterial colonies.

For application in the environment of livestock animals, it is advantageous that the composition be dispersed or spread at a rate of 10⁵ to 10¹¹ bacterial colonies for a dry or dehydrated composition, or 10⁵ to 10¹¹ bacterial colonies for a liquid composition per m² of surface or medium to be treated.

Also advantageously, the invention relates to a composition for its above-mentioned use, wherein the environment is cleaned and disinfected, and the terrestrial animals are washed prior to the composition being spread in the rearing environment of said terrestrial animals and/or applied to said terrestrial animals.

It may be advantageous to clean the environment and/or animals before using the composition according to the invention. Such cleaning may be assimilated to a disinfection. Before implantation of the flora of the composition according to the invention, if cleaning or disinfection is carried out, the better the efficiency of the bacteria will be, and the better the benefit for the animals. Surfaces may be treated by any cleaning means known to those skilled in the art. As far as the cleaning of animals is concerned, they can be washed, bathed or even shampooed.

The invention further relates to a kit intended for terrestrial livestock animals against pathogenic infections affecting the health and well-being of livestock animals comprising
a composition as defined in any of the preceding claims, and
at least one biosafety agent and/or at least one biocontrol agent.

In the invention, a biosafety agent means a chemical or physical agent capable of reducing the number or inactivating microorganisms and their biofilms. Examples include disinfectants, detergents, UV radiation, or water vapor or other biocidal agents. This list is not exhaustive, and the person skilled in the art will be aware of biosafety agents.

In the invention, a biocontrol agent is understood to mean any composition of living organisms or derived from living organisms, or composition of natural substances intended to protect animals from a sanitary point of view (macro-organisms (insects, nematodes, etc.), microorganisms (viruses, bacteria, phages or fungi), chemical mediators (pheromones), or natural substances of mineral, plant or animal origin, such as essential oils).

In the invention, it is proposed to provide a kit which comprises the composition as defined above, and in particular a composition comprising the bacteria NOL01, NOL02, and NOL03 with the bacterium NOL11, and in parallel at least one biocontrol and/or biosafety agent.

This kit provides the user with the means to apply the composition according to the invention, and where appropriate, before or after treatment with the composition according to the invention, to use one or more other agents as defined above.

It should be noted that in order to maintain the effect of the composition according to the invention, the above-mentioned agent must not be used simultaneously with the composition according to the invention if it has an antibacterial effect.

The invention will be better understood in the light of the three examples and the 13 figures.

### EXAMPLES

### Example 1

### Application in the Environment: Bedding of Farmed Calves

In this first example, the inventors seek to show whether an application of the composition according to the invention in the environment or on the bedding, alone, is of benefit for the prevention of pathogenic infections.

This trial was carried out on calves from a Holstein dairy cow farm in France in the department of Ille-et-Vilaine.

The product trialed was a combination of live bacteria (strains of Bacillus subtilis and Lactococcus lactis) in their culture medium, brought frozen to the farm, to be diluted in water before spraying.

The trial was carried out in the calf house with a control batch and a batch where the product was sprayed in the environment and on the bedding. The impact of the product was studied over time.

The influence of the product was measured on the chemical qualities of the bedding, the cleanliness of the animals and the health balance of the batch (frequency and nature of pathologies, veterinary expenses) over time.

Equipment and Methods

a—Animals

16 Holstein breeding calves from 6 weeks to 5 and a half months old, divided into groups of 3, 4 or 5 according to their age in 4 pens of 15 m² straw area, the first two pens being separated from the other two by a wall.

b—Product Used and Equipment

One dose of the composition was packaged in two separate 20 ml vials:
vial B containing bacillus (NOL01, NOL02, NOL03)
vial L containing Lactococcus (NOL11)

The vials were brought frozen to the farm.

The water used for spraying must not contain chlorine. Pre-treatment of the water with sodium thiosulphate may therefore be necessary.

The equipment required for the treatment and measurements was as follows:
8 boxes of 2 vials of composition
sodium thiosulfate
1 sprayer
1 bovine tape

c—Experimental Device

Prior to the Start of the Trials:
Care should be taken to check that the sprayer is working properly.
Total surface area of straw area (control+trial): 60 m², i.e. approximately 4 m² per animal.
Surface treated with the composition according to the invention: 2 boxes of 15 m².
Control surface: 2 boxes of 15 m².

Separation of control pens and trial pens by a wall.

The weight of the calves was estimated at the start of the trial using a bovine tape measure. The distribution of calves in each pen was carried out to obtain a comparable total live weight between control pens and trial pens.

d—Process of the Trial and Measurements

The trial lasted 2 months, the period between two cleanings.

TABLE 1

| | Wk | Wk | Wk | Wk | Wk | Wk | Wk | Wk | Wk |
|---|---|---|---|---|---|---|---|---|---|
| | 16 | 17 | 18 | 19 | 20 | 21 | 22 | 23 | 24 |
| Cleaning | x | | | | | | | | x |
| Spraying of pens | X | X | X | X | X | X | X | X | |
| 3 and 4 | | | | | | | | | |

The first application of the product was carried out after the cleaning in week 16. The first measurements were taken just before the first application of the flora. Then the measurements were taken every week before application of the product. The product applications ended in week 23, and the last measurement was taken just before the second cleaning in week 24.

e—Application

Directions for use: to be applied in liquid form. One dose of product (consisting of vials B and L) is required for each weekly spraying.

1 L of diluted composition was used to treat the 30 m² of the calf trial.

Procedure for Preparing the Solution:
Let the product thaw at room temperature between 15 and 25° C. until complete liquefaction (approx. 1 hour±15 minutes).
Prepare 10 L of water and add 160 mg of sodium thiosulphate (if water is treated with chlorine, add sodium thiosulphate at a rate of 16 mg/L of water. If the water is treated with hydrogen peroxide, use mineral water or untreated water).
Remove the cap and remove the stopper from the vials.
Pour the two vials of the product into the treated water.
Mix to homogenize the product and let stand for 15 minutes.
In a sprayer which has not contained a disinfectant, pour 1 L of the composition according to the invention, said composition being diluted, and spray on the bedding and all the surfaces of pens 3 and 4.
This application was renewed every week of the trial (8 in total).

During the trial, no disinfection was carried out in the building.

f—Mulching Practices

The breeder shall maintain his usual mulching practices for the duration of the trial.
Introduce 1 round of straw for 30 m² after cleaning.
Mulching 2 to 3 times a week
Distribute the quantities of straw equally in each of the pens.
On measurement/treatment days, mulching is carried out after these practices.

g—Measurements and Analyses

Date of Sampling and Measurements:

TABLE 2

| | Wk | Wk | Wk | Wk | Wk | Wk | Wk | Wk | Wk |
|---|---|---|---|---|---|---|---|---|---|
| | 16 | 17 | 18 | 19 | 20 | 21 | 22 | 23 | 24 |
| Cleaning | x | | | | | | | | x |
| DM of the | x | | x | | x | | x | | x |
| bedding | | | | | | | | | |
| T° of the | x | x | x | x | x | x | x | x | x |
| bedding | | | | | | | | | |
| Height of | x | x | x | x | x | x | x | x | x |
| bedding | | | | | | | | | |

Temperature Measurements

Take a weekly T° measurement at 0-20 cm from the first cleaning at the surface of the straw and at depth (indicate the depth of the first measurement to make the same throughout the trial; if the thickness increases, a third deeper measurement can be taken (total length of the probe).
DM Measurements

From the first cleaning, once every two weeks, take samples of bedding (3-4 handfuls for a total of 200 g minimum) at 0-20 cm, and measure the DM (send samples to the laboratory). In the weeks with cleaning, take the measurement on the day of cleaning, just before the start of the procedure.

For each sampling, take a sample at all sampling points and collect the samples by pen. This will give 4 samples of at least 1 kg. Homogenize before sending for analysis and identify as follows: pen number (1, 2, 3 and 4)/date of sampling/analysis (DM)
Measurements on Animals

Health: ask the farmer to note any health problems that appear during the trial+the veterinary fees for each period (diarrhea, respiratory or other problems).

Results and Conclusion:

The results of bedding temperature and dry matter were equivalent between the two batches.

Some diarrhea was observed, but no significant difference between the control batch and the trial batch (treated with the composition according to the invention), particularly in respect of the quality of the feces and in terms of the cleanliness of the pens, where the results were equivalent between the batches.

Therefore, application in the environment alone is not sufficient to improve the living conditions of the animals.

### Example 2

### Application to the Animal—Poultry

Background and Objectives

In this second example the objective was to determine if the application to the animal can have a better effect. The objective of this trial was to test the application of the barrier flora (frozen combination of Bacillus and Lactococcus) from the hatchery, to the chicks at D0, in order to study its impact on the health quality of the batches. The trial was carried out on chicks destined for 20 farms, which were compared with contemporary control batches. In each test farm, only chicks from one building were treated in the hatchery; chicks from the second building were not treated (control).

The aim was to verify an effect on the reduction of mortality, the improvement of health and the improvement of zootechnical indices.

The composition according to the invention (strains of Bacillus subtilis NOL01, NOL02, NOL03 and of Lactococcus lactis NOL03) was applied directly to the chicks after sexing. The doses were thawed in a water bath of 27° C. for 9 minutes 30.

They were then diluted in 3 L of mineral water for 30,000 chicks, i.e. 0.1 mL per chick. This recommendation takes into account the volume of water used for spray vaccination, i.e. 0.2 to 0.3 mL.

100 males and 100 females were weighed at D7 in each building.

The parameters studied were: food and water consumption, mortality and elimination as well as slaughterhouse data (technical-economic results, seizures, counting of pododermatitis, tarsi, infected skin lesions or ISLs and sternums).

Results:

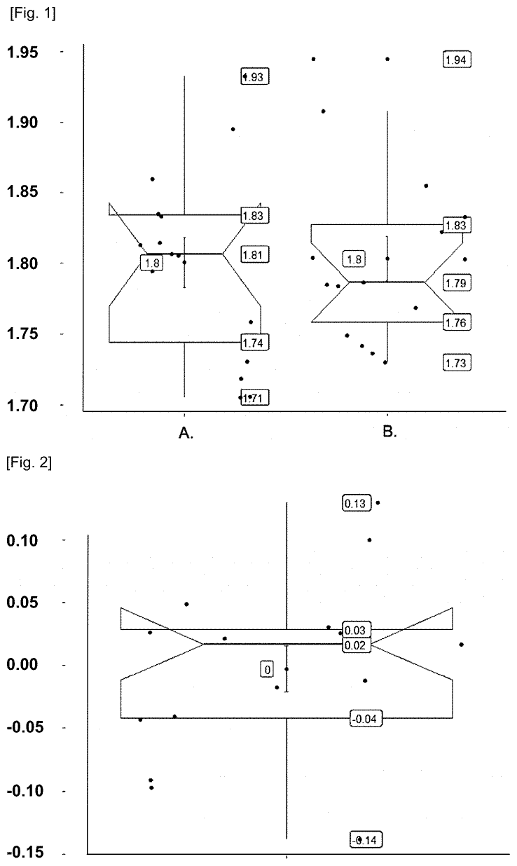

The results observed for the corrected consumption index (CCI) are shown in FIGS. 1 and 2.

With a Student test: p-value=0.882, the difference between treated and untreated animals is not significant.

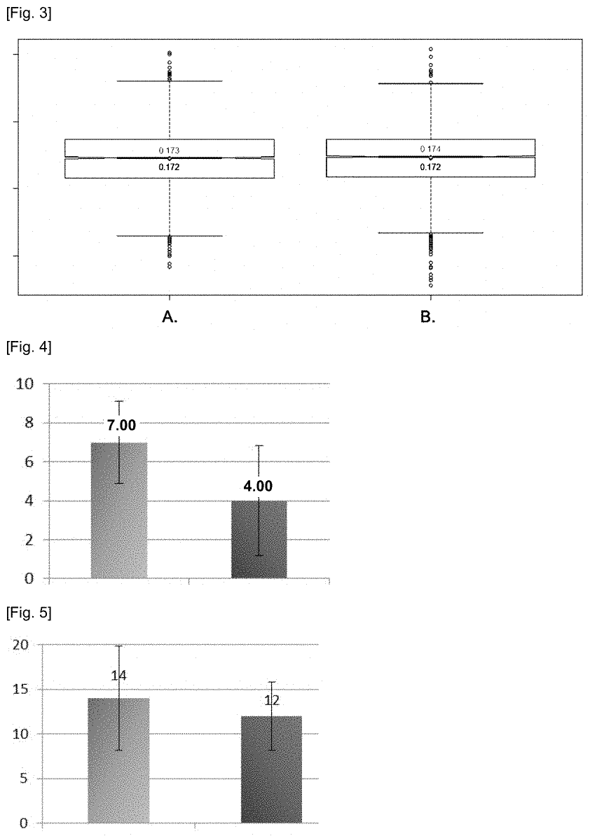

The same was true for the chick feed margin (CFM), burned tarsi, mortality, antibiotic treatments, weights of the animals mentioned in FIG. 3, and pododermatitis, which were not significantly different between the controls and the animals having received the composition according to the invention.

Thus, it can be seen that application to the animal alone does not benefit the animal.

### Example 3

### Application in the Environment, and on the Animal: Poultry

Objective

The purpose of this trial was to test the efficacy of the composition according to the invention (NOL01, NIL02, NOL03, NOL11) both in the animal and in the environment. The aim was to identify an optimal use for the prevention of pathologies (lameness, colibacillosis, etc.) and/or intestinal health and the improvement of the quality of the bedding and therefore of the animal's well-being.

Test System

2 farms tested.

The first farm tested had 2 comparable buildings, with buckwheat husk bedding on concrete floor. It was equipped with weighing scales. Its drinking water was treated with 3% pure hypochlorous acid at 80% (electrolysis treatment system—DCW company).

The second farm had 2 comparable buildings, with buckwheat husk bedding and wood shavings on a non-concrete floor. It was equipped with weighing scales. Its drinking water was treated with hydrogen peroxide, stopping the treatment when the flora was sprayed.

The influence of the product was measured on growth performance, the health assessment of the batch (frequency and nature of pathologies, veterinary costs), bacteriological counts (bedding), gas emissions, bedding quality and well-being criteria.

Application

Application of 5 doses during rearing.

5 doses distributed as follows: D0-D3-D7-D9-D14-D21.

Seeding on D0 was carried out after setting up.

Spray the environment and animals.

The trial and control buildings were distinguished only by whether or not the barrier flora was applied. The product was sprayed by fogging (about 15-20 L of water containing the flora at each application, for 1200 m²). The water used was untreated artesian well water.

Results:

The following table shows a comparison of the results between the control and trial buildings for the two farms.

TABLE 3

| | Rearing 1 | Rearing 2 |
|---|---|---|
| Zootechnical | | |
| performances | | |
| ICC | +4 pts | +2.5 pts |
| Well-being | | |
| Water/Food | +before 28 d | − |
| | −after 28 d | |
| Bedding | −before 28 d | −at the end of the flock |
| Dry Matter | −except at the end of the | − |
| | flock | |
| Pododermatites | − | + |
| Health status | | |
| Treatments | more treatments in the | several treatments in both |
| | control batch | batches |
| Mortality | +for males | +for females before 27 d then |
| | | −−for males after 27 d |
| Sorting | +for males | − |
| Standing on legs | + | − |
| Seizures | +for females | − |
| Bacteriology | | |
| Bedding | No significant difference | No significant difference |

Conclusions:

Animal and environmental applications alone are not sufficient to improve animal well-being. Indeed, the results on the 2 farms show contradictory effects on lameness, mortality and pododermatitis, no significant difference on the bacteriology of the bedding and a degraded feed intake index.

### Example 4

### Application in the Environment, on the Animal and Ingested: Poultry

The purpose of this trial was to determine whether a combination of environmental, animal and ingestion applications of the composition described in Example 1 is effective in preventing pathogenic infections.

1. Test System

A preliminary statistical study made it possible to classify the meat poultry farms according to the proximity of the zootechnical results of two farm buildings. This classification included all farms with at least 5 flocks since the beginning of 2014 for which weight, mortality, CCI and pododermatitis values were provided. This study enabled a list to be drawn up of the 60 farms with the closest results between two buildings.

The trial therefore took place in about 20 farms on this list, with one control and one trial building receiving an application of barrier flora in each test farm. Prior to the integration of each farm in the trial, it was ensured that the two trial buildings would receive the same lines from the same hatchery for 2 flocks.

1. Product Used and Equipment

The product was applied with battery-powered sprayers or with farmers' sprayers used for vaccination (after validation of the equipment: sufficient capacity, practicality, no use for disinfection treatments or any other incompatible product).

The equipment required for treatment and measurements was as follows:

Boxes of 2 vials of flora

Mineral water

Battery-powered sprayer

Gloves

Boot covers

Overalls

1. Applications

Two types of application were realized: in the building+animals and in the water circuit.

Environment and Animal Applications:

Spraying on: bedding, feeders, drinkers and animals.

1 dose for 1000 to 1200 m² or 1 dose for 20 to 24,000 chicks.

Application: D0 (just after setting up), D10 and D20.

Procedure for Preparing the Solution:

Allow the product to thaw at room temperature between 15 and 25° C. until complete liquefaction (approx. 1 hour±15 minutes) or 5 minutes in a 30° C. water bath.

Pour 10 L of water into the spray tank (if water is treated with chlorine, add sodium thiosulphate to 16 mg/L of water. If water is treated with hydrogen peroxide, use mineral water or untreated water).

Remove the cap and remove the stopper from the vials.

Pour both vials of the product into water (treated or mineral).

Stir to homogenize the product and let stand for 15 minutes before starting spraying. Spray the solution throughout the building and on animals.

Application in the Water Circuit:

Stop water treatment (or neutralization with sodium thiosulfate if the water is chlorinated).

1 dose for 20 to 24,000 chicks.

Application to: D0-D1-D2-D10-D20-D30-D40

Procedure for Preparing the Solution:

Allow the product to thaw at room temperature between 15 and 25° C. until complete liquefaction (approx. 1 hour±15 minutes) or 5 minutes in a 30° C. water bath.

Fill the tank with approximately 100 L of water for applications up to D10 and 300 L for others (if water is treated with chlorine, add sodium thiosulphate to 16 mg/L of water. If other water treatment is done, stop on treatment days).

Remove the cap and remove the stopper from the vials.

Pour the two vials of the product into the water.

Mixing to homogenize the product.

If an antibiotic treatment is carried out when the chicks arrive, postpone treatment of the water circuit to two days after the end of the antibiotic.

1. Results

FIGS. 4 to 10 show the results obtained for lameness, mortality, antibiotic treatment, burned tarsi in males, and food consumption.

FIGS. 4 and 5 show a significant decrease in the number of animals with burned tarsi in males (FIG. 4) or females (FIG. 5) during the triple treatment with the composition according to the invention, compared to the control sample.

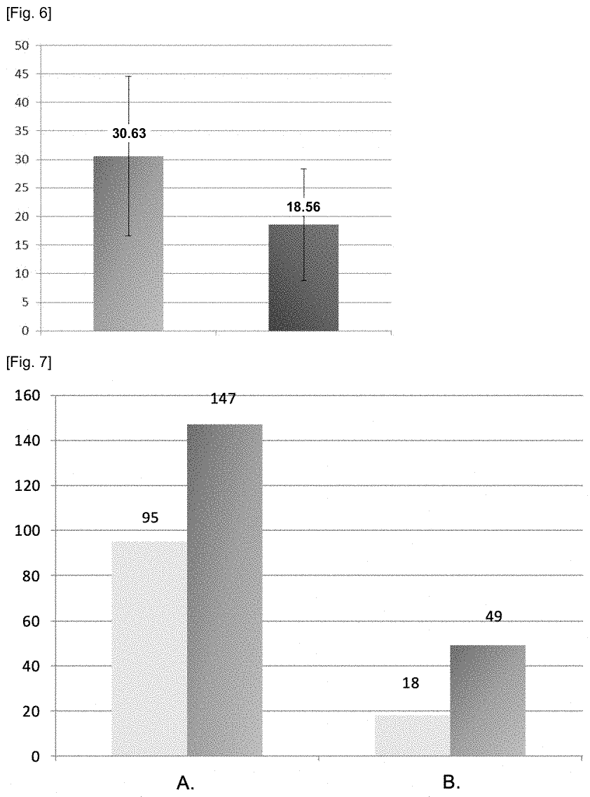

FIGS. 6 and 7 show that animals treated with the composition according to the invention in all three modes of application required significantly less antibiotics. There is a trend of 12 ppm/kg live weight less antibiotic administered in the trial batches compared to controls, i.e. a 39% decrease in antibiotic consumption.

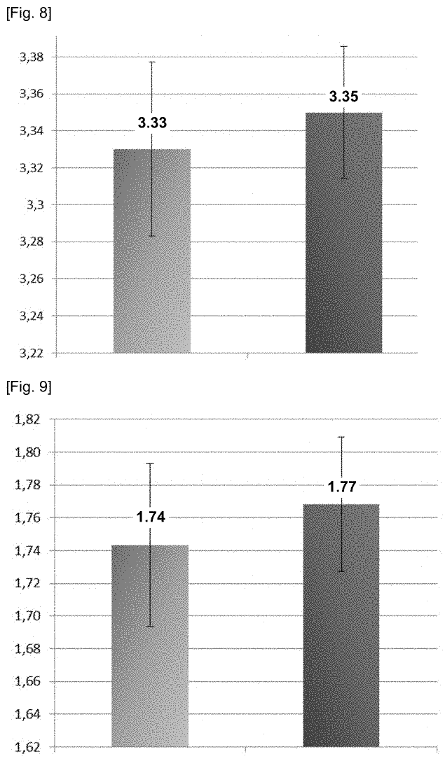

The males had a higher weight after treatment with the composition according to the invention (FIG. 8), as did the females before the first slaughter (FIG. 9).

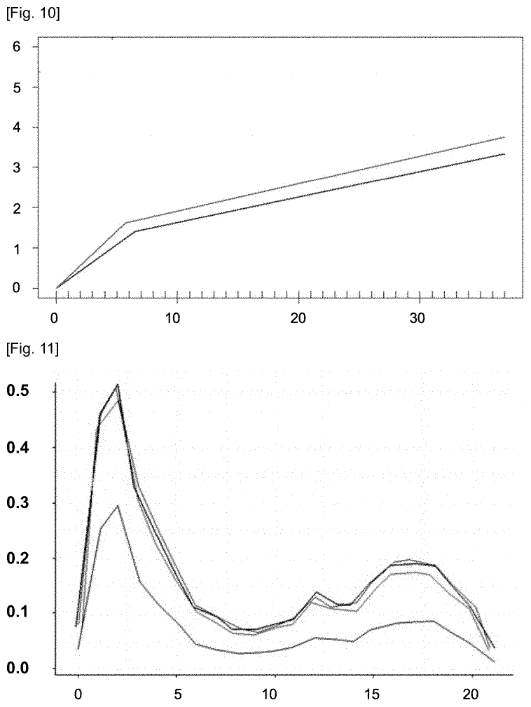

Finally, mortality was reduced in animals having been treated with the compositions according to the invention compared to control animals (FIG. 10).

In conclusion, there is a significant benefit to animal health and well-being when the composition is used simultaneously in the environment, applied to the animal and ingested via drinking water.

### Example 5

### Application in the Environment, to the Animal and by Inhalation: Pigs

The purpose of this trial was to show whether a combined application of the composition described in Example 1 in the environment, on the animal and by inhalation was effective. Therefore, check in relation to the previous example whether other types of applications can be carried out such as inhalation instead of ingestion.

The trial was carried out in 2 different farms according to the following protocols.

Farm 1:

TABLE 4

| | | Control treatment | Trial treatment | Trial treatment |
|---|---|---|---|---|
| Stage | Event | (placebo) | PS | PS + ENG |
| PW + FAT | Empty underfloor | Yes | Yes | Yes |
| | gutters, disinfection | | | |
| | cleaning (DC) | | | |
| | then disinfection | | | |
| | downtime (DD) | | | |
| PW | No. of pigs per pen | 180 | 180 | 180 |
| | Samp* D0 | Yes | Yes | Yes |
| | Spr* D0 | Placebo | 2 doses/1000 m² | 2 doses/1000 m² |
| | Spr D15 | Placebo | 1 dose/1000 m² | 1 dose/1000 m² |
| | Spr D30 | Placebo | 1 dose/1000 m² | 1 dose/1000 m² |
| | Samp D42 | Yes | Yes | Yes |
| FAT | Samp D0 | Yes | Yes | Yes |
| | Spr D0 | Placebo | Placebo | 1 dose/1000 m² |
| | Spr D15 | Placebo | Placebo | 1 dose/1000 m² |
| | Spr D45 | Placebo | Placebo | 1 dose/1000 m² |
| | Samp D50 | Yes | Yes | Yes |
| | Spr D75 | Placebo | Placebo | 1 dose/1000 m² |
| | Samp D90 | Yes | Yes | Yes |

*Samp = Bacteriological samples, Spr = spraying, FAT: fattening, PW: post weaning

Farm 2:

TABLE 5

| | | Control treatment | Trial treatment |
|---|---|---|---|
| Stage | Event | (placebo) | PW + FAT |
| PW + FAT | Empty underfloor | Yes | Yes |
| | gutters, DC then DD | | |
| PW | No. of pigs per pen | 350 | 350 |
| | Samp* D0 | Yes | Yes |
| | Spr* D0 | Placebo | 2 doses/1000 m² |
| | Spr D15 | Placebo | 1 dose/1000 m² |
| | Spr D30 | Placebo | 1 dose/1000 m² |
| | Samp D42 | Yes | Yes |
| ENG | Samp D0 | Yes | Yes |
| | Spr D0 | Placebo | 1 dose/1000 m² |
| | Spr D15 | Placebo | 1 dose/1000 m² |
| | Spr D45 | Placebo | 1 dose/1000 m² |
| | Samp D50 | Yes | Yes |
| | Spr D75 | Placebo | 1 dose/1000 m² |
| | Samp D90 | Yes | Yes |
| SLA | Slaughter J100 | Yes | Yes |
| | (snouts) | | |

*Samp = Bacteriological samples, Spr = spraying, FAT: Fattening, PW: Post-weaning, SLA: Slaughter

In this example, animals treated with the composition according to the invention according to the three modes of treatment were compared with animals not having received the composition of the invention (placebo).

The application was carried out with a sprayer in the presence of the animals so the flora were applied on the surfaces of the pens (E), on the animals (A) and were inhaled (I).

Pigs randomly taken from farm 2 were analyzed after slaughter by measuring deformations on snout sections.

Different bacteriological samples were taken before the first application of flora in the different pens to evaluate the quality of the cleaning and disinfection step and at the exit of the animals from the pens after the different applications of flora to evaluate the pathogenic pressure.

Results:

The results are compiled in the following table:

TABLE 6

| Nose scoring | Farm 2 | |
|---|---|---|
| (snout section in | Placebo | Flora |
| slaughterhouse) | n = 20 PC | n = 19 PC |
| Score 0 | 6 | 12 |
| Score 1 | 10 | 7 |
| Score 2 | 3 | 0 |
| Score 3 | 1 | 0 |
| Score 4 | 0 | 0 |
| Average | 0.95 | 0.37 |
| nasal condition VG | 30% | 63% |

nasal condition VG: very good nasal condition

Score 0: no deviation of the septum or atrophy of the scrolls and conchae

Score 4: deviation of the septum and atrophy of the scrolls and conchae

The placebo sample (without composition) indicates 30% of the individuals with a very good nasal anatomical condition (absence of deviation of the nasal septum, absence of atrophy of the nasal scrolls and conchae).

The trial sample (pigs reared in the presence of barrier flora from post-weaning entry to the end of rearing) revealed that twice as many (63%) individuals recorded a very good nasal anatomical condition vs. placebo.

It appears that the application according to the invention has a significant effect on the health and well-being of animals.

The bacteriological results show that the better the cleaning and disinfection phase of the pens was carried out, the better the barrier flora showed better results on the inhibition of the pathogenic flora:

TABLE 7

| | | | | |
|---|---|---|---|---|
| Score of the quality | Score 1.3 | Score 1.9 | Score 2.2 | Score 2.4 |
| of the disinfection | | | | |
| cleaning phase | | | | |
| (1 = excellent to | | | | |
| 4 = absent) | | | | |
| Frequency of best | 5/6 (83%) | 4/6 (67%) | 3/6 (50%) | 0/6 (0%) |
| barrier flora | | | | |
| result on pathogenic | | | | |
| flora vs. | | | | |
| placebo, at the end | | | | |
| of the period | | | | |

Conclusions

This trial thus shows the importance of environment application, the effectiveness of which is improved when the cleaning and disinfection of surfaces prior to application is properly carried out. The benefit of the product can be measured both on the animals (health), and with regard to the farmer (working conditions).

### Example 6

### Application in the Environment and on the Animal Versus Environmental, Animal and Ingested Application: Pigs

The purpose of this trial was to confirm the benefit of combining the three modes of application of the composition described in Example 1 compared to the combination of only two of them.

The farm monitored was a farm of 700 breeder-fattener sows with multiplication selection (5 groups of 120 sows). The trial was carried out on 2 groups, within which 3 batches of 40 sows were formed, maintaining a homogeneous distribution of ages. The application protocol included the spraying of a barrier flora in the environment, on the udder and perineum of the sows as well as probiotic administration to the sows via the feeder.

Spraying Protocols Applied

TABLE 8

| | Batch 1 | Batch 2 | Batch 3 | Batch 4 |
|---|---|---|---|---|
| Group | 1 + 2 | 1 | 1 + 2 | 2 |
| Antibiotic | yes | yes | yes | no |
| on piglets | | | | |
| D-3 | Environment | Environment | Environment | Environment |
| | and sows | and sows | and sows | and sows |
| D-2 | | | | |
| D-1 | Udders and | Udders and | Udders and | Udders and |
| | perineum | perineum | perineum | perineum |
| D0: | | | | |
| Farrowing | | | | |
| D1 | Udders and | Udders and | Udders and | Udders and |
| | perineum | perineum | perineum | perineum |
| D4 | Udders and | Udders and | | |
| | perineum | perineum | | |
| Protocol | A + E | A + E | A + E | A + E + I |
| equivalence | | | | |

All sows received a dose of probiotics at trough from D-3 to D1 based on the time of farrowing.

In the second group, all sows were shampooed before arrival in the maternity ward, unlike the first group.

Only piglets in batch 4 did not receive antibiotics at the time of care. As the antibiotic used for all the other batches was very effective against the positive flora, the inventors consider in this trial that the protocol of these batches is equivalent to a purely environmental and animal application because the flora ingested by the piglets did not survive the antibiotic.

The inventors are therefore interested here in the differences observed between batches 1 to 3 (A+E) with batch 4 (A+E+I).

Results:

FIGS. 11 and 12 show that with the three applications combined the risk of diarrhea is lower and the risk of mortality is also lower compared to an environmental and animal only application.

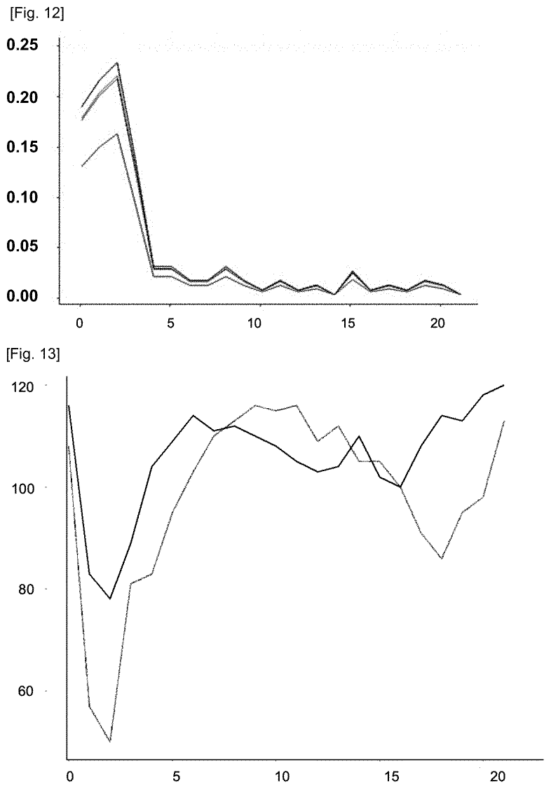

FIG. 13 shows that more pens with piglets whose mothers had been shampooed prior to the application of flora are digestively healthy compared to pens with piglets whose sows had not been shampooed. Indeed, in the first case (group 2), 78 pens with normal feces out of 120 were observed at the lowest level, whereas in the second case (group 1), only 50 pens out of 120 were observed.

This result also shows the importance of the application on the animal, preferably by the shampooing of sows for a better development of positive flora, to increase the efficiency of the product.

Conclusions

In this example, the inventors have shown that the A+E+I application is more efficient than the A+E application. Moreover, they showed that if we favor the implantation of the flora on the animal (application A best achieved), we improve the prevention of diarrhea.

The invention is not limited to the embodiments presented, and other embodiments will clearly be clear to a person skilled in the art.

1-10. (canceled)

11. A method for the prevention or treatment of pathogenic infections affecting the health of terrestrial livestock animals, said method comprising:
a first step of administrating to terrestrial livestock animals, by ingestion or inhalation, a bacterial composition comprising at least one bacterial strain of the genus Bacillus and at least one strain of lactic bacteria, and
a second step of:
(a1) spreading the composition within the rearing environment of said terrestrial livestock animals,
(a2) applying the composition to said terrestrial livestock animals, or
(a3) spreading the composition within the rearing environment of said terrestrial livestock animals and applied to said terrestrial livestock animals.

12. The method according to claim 11, wherein the bacterial composition comprises:
at least one of the following three strains of Bacillus subtilis: NOL01, NOL02, NOL03, said strains being deposited, respectively, at the CNCM under the numbers CNCM I-4606, CNCM I-5043 and CNCM I-4607, and
at least the strain of lactic bacteria: Lactococcus lactis spp lactis 1 strain NOL11, said strain being deposited at the CNCM under the number CNCM I-4609.

13. The method according to claim 11, wherein the bacterial composition comprises from 10⁴ to 10¹¹ bacterial colonies of Bacillus and from 10⁴ to 10¹¹ bacterial colonies of lactic bacteria, the bacterial colonies being in grams or milliliters of composition.

14. The method of claim 11, wherein said at least one strain of Bacillus is in sporulated and/or vegetative form.

15. The method according to claim 11, wherein the first and the second step are carried out simultaneously, separately or staggered over time.

16. The method according to claim 11, wherein said second step is carried out prior the first step.

17. The method according to claim 11, wherein the bacterial composition consists essentially of the four bacterial strains from the group consisting of the three Bacillus subtilis strains NOL01, NOL02, NOL03, said strains being deposited, respectively, at the CNCM under numbers CNCM I-4606, CNCM I-5043 and CNCM I-4607, and the Lactococcus lactis spp lactis 1 strain NOL11, said strain being deposited at the CNCM under number CNCM I-4609.

18. The method according to claim 11, wherein the bacterial composition is spread per m² at a rate of 10⁵ to 10¹¹ bacterial colonies.

19. The method according to claim 11, wherein the environment is cleaned and disinfected and the terrestrial animals are washed prior to the spreading in the rearing environment of said terrestrial livestock animals and/or the application on said terrestrial livestock animals.

20. A kit for the prevention of pathogenic infections affecting the health and well-being of terrestrial livestock animals comprising:
a composition as defined in claim 11, and
at least one biosafety agent and/or at least one biocontrol agent.
